(19) United States
(12) Patent Application Publication
(10) Pub. No.: US 2021/0015418 A1
(43) Pub. Date: Jan. 21, 2021

(54) INFORMATION PROCESSING SYSTEM, INFORMATION PROCESSING APPARATUS, AND INFORMATION PROCESSING METHOD

(71) Applicant: SONY CORPORATION, TOKYO (JP)

(72) Inventor: MASAYOSHI MORITA, TOKYO (JP)

(21) Appl. No.: 17/040,048

(22) PCT Filed: Feb. 21, 2019
(86) PCT No.: PCT/JP2019/006578

§ 371 Date: Sep. 21, 2020

(30) Foreign Application Priority Data
Mar. 29, 2018 (JP) 2018-063626

(51) Int. Cl. A61B 5/16; A61B 5/11; A61B 5/00

(52) U.S. Cl. CPC A61B 5/165; A61B 5/1126; A61B 5/6831; A61B 2562/0247; A61B 5/6803; A61B 5/681; A61B 5/7203

(57) ABSTRACT

[Overview] [Problem to be Solved] To grasp a contact pressure on a first sensor with a surface of a living body and reduce its influence on the quality of a signal obtained by the first sensor.

[Solution] There is provided an information processing system including: a sensor section including a first sensor that detects information for judging an emotion of a living body and a second sensor that detects a body motion pressure of a region of the living body corresponding to a detection region of the first sensor; and a correction processing section that corrects first sensor information obtained by the first sensor on the basis of second sensor information obtained by the second sensor. This makes it possible to grasp a contact pressure on the first sensor with a surface of the living body and reduce its influence on the quality of a signal obtained by the first sensor.

### TECHNICAL FIELD

The present disclosure relates to an information processing system, an information processing apparatus, and an information processing method.

### BACKGROUND ART

Measurement technique intended for living bodies has been developed in recent years. For example, PTL 1, etc. disclose a technique to detect the pulse wave of a living body. In the technique disclosed in PTL 1, in a state where an air bag that contains air is half-pressed against the living body by an air pressing device, a pressure sensor detects a pressing force from the living body. By detecting this pressing force, for example, the pulse wave or blood pressure, is detected. Besides this, various techniques to measure the heartbeat, body temperature, etc. have been developed as measurement technique intended for living bodies.

In a case of sensing biological information by means of a biosensor device intended for a living body as a measurement object like those described in PTL 1, etc., a contact pressure between the biosensor device and the living body is known to greatly affect the quality of a signal acquired by a biosensor.

### CITATION LIST

### Patent Literature

PTL 1: Japanese Unexamined Patent Application Publication No. H02-1224

### SUMMARY OF THE INVENTION

In the technique described in PTL 1, etc., a pressing force applied to the living body is ensured by the air pressing device. However, the air pressing device is likely to increase in volume; therefore, in a case where the air pressing device is provided in a biosensor attached to a living body, it may impede the motion of the living body with the biosensor attached.

Accordingly, there has been required a technique that makes it possible to improve the signal quality of information acquired by a biosensor without having to use an air pressing device or something like the one described above.

In view of the above-described situation, it is desirable to grasp a contact pressure on the biosensor with the surface of the living body and reduce its influence on the signal quality.

According to the present disclosure, there is provided an information processing system including: a sensor section including a first sensor that detects information for judging an emotion of a living body and a second sensor that detects a body motion pressure of a region of the living body corresponding to a detection region of the first sensor; and a correction processing section that corrects first sensor information obtained by the first sensor on the basis of second sensor information obtained by the second sensor.

Furthermore, according to the present disclosure, there is provided an information processing apparatus including: a sensor section including a first sensor that detects information for judging an emotion of a living body and a second sensor that detects a body motion pressure of a region of the living body corresponding to a detection region of the first sensor; and a correction processing section that corrects first sensor information obtained by the first sensor on the basis of second sensor information obtained by the second sensor.

Moreover, according to the present disclosure, there is provided an information processing method implemented by a processor, the information processing method including: acquiring respective pieces of information detected by a first sensor and a second sensor, the first sensor detecting information for judging an emotion of a living body, the second sensor detecting a body motion pressure of a region of the living body corresponding to a detection region of the first sensor; and correcting first sensor information obtained by the first sensor on the basis of second sensor information obtained by the second sensor.

According to the present disclosure, it is possible for the second sensor to detect the body motion pressure of the region corresponding to the region subjected to the detection by the first sensor; therefore, it is possible to correct the information detected by the first sensor on the basis of the body motion pressure of the corresponding region.

As described above, according to the present disclosure, it is possible to improve the quality of first sensor information.

It is to be noted that the above-described effects are not necessarily limitative, and any of effects described in the present specification or other effects that may be understandable from the present specification may be achieved in addition to the above-described effects or instead of the above-described effects.

### BRIEF DESCRIPTION OF DRAWINGS

FIG. 1 is a block diagram illustrating an internal configuration of an information processing system according to an embodiment of the present disclosure.

FIG. 2A is a diagram illustrating an example of the attachment of the information processing system according to the embodiment to a living body.

FIG. 2B is a diagram illustrating an example of the attachment of the information processing system according to the embodiment to the living body.

FIG. 3 is a diagram illustrating an example of the appearance of the information processing system according to the embodiment.

FIG. 4A is a schematic diagram illustrating an example of an external configuration of the information processing system according to the embodiment.

FIG. 4B is a schematic diagram illustrating the example of the external configuration of the information processing system according to the embodiment.

FIG. 5A is a diagram illustrating an example of the detailed external configuration of the information processing system according to the embodiment.

FIG. 5B is a diagram illustrating the example of the detailed external configuration of the information processing system according to the embodiment.

FIG. 6 is a diagram illustrating an example of the operation flow of the information processing system according to the embodiment.

FIG. 7 is a block diagram illustrating a hardware configuration example of an information processing system according to an embodiment of the present disclosure.

### MODES FOR CARRYING OUT THE INVENTION

In the following, a preferred embodiment of the present disclosure is described in detail with reference to accompanying drawings. It is to be noted that in the present specification and the drawings, components having substantially the same functional configuration are assigned the same reference numeral to avoid the repetition of description.

It is to be noted that description is given in the following order.

### 1. Appearance of Information Processing System

### 2. Internal Configuration of Information Processing System

### 3. External Configuration of Information Processing System

3.1. Outline of Configuration

3.2. Details of Configuration

### 4. Operation Flow of Information Processing System

### 5. Hardware Configuration Example

### (1. Appearance of Information Processing System)

An information processing system of the present embodiment is a system that detects information regarding a state of a living body and judges an emotion of the living body on the basis of the detected information. The information processing system of the present embodiment may be directly attached to the living body to detect the information regarding the state of the living body.

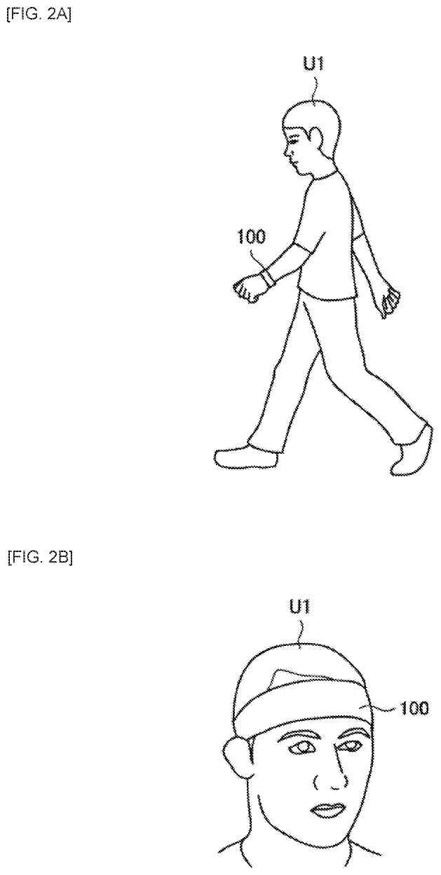

Specifically, to judge an emotion of the living body, the information processing system of the present embodiment is used, for example, as illustrated in FIG. 2A or 2B. FIGS. 2A and 2B are diagrams illustrating a state of the information processing system of the present embodiment attached to the living body. In FIG. 2A, a user U1 has a wristwatch-type information processing system 100 with a strap worn on his left wrist. In FIG. 2B, the user U1 has a headband-type information processing system 100 worn around his head. These information processing systems 100 detect information for judging an emotion of the living body, such as a sweat, pulse wave, myoelectricity, blood pressure, or body temperature of the user U1, and grasp biological information of the user U1. On the basis of this biological information, the information processing system 100 is able to check the user's state, such as if he is concentrating or if he is awake.

FIGS. 2A and 2B illustrate examples where the information processing system 100 is attached to the arm or the head; however, the information processing system 100 is not limited to such examples. For example, the information processing system 100 may be realized in a form that is wearable on a part of the living body such as the user's hand, such as a wristband, a glove, a smartwatch, or a finger ring. Furthermore, for example, the information processing system 100 may have a form of being included in an object that the user may touch. Specifically, the information processing system 100 may be provided on the surface of or inside an object that the user may touch, such as a mobile phone, a smartphone, a tablet, a mouse, a keyboard, a handle, a lever, a camera, sporting equipment (a golf club, a tennis racket, the grip of an archery bow, etc.), or a writing implement.

Moreover, the information processing system 100 may be realized in a form that is wearable on a portion of the user's head, for example, such as a hat, an accessory, goggles, or glasses. Furthermore, the information processing system 100 may be provided in clothes such as sportswear, socks, protective equipment, shoes, etc.

That is, the form in which the information processing system 100 is realized is not particularly limited, and the information processing system 100 may be realized in any form as long as it is provided to be able to come in contact with the surface of the living body. Furthermore, the information processing system 100 does not have to be in direct contact with the body surface of the living body as long as it is able to detect information regarding a state of the living body. For example, the information processing system 100 may be in contact with the surface of the living body through, for example, clothes or a protective film.

Furthermore, the information processing system 100 may be a system that judges an emotion of the living body by causing another device to perform information processing on the basis of information detected by a sensor in contact with the living body. For example, in a case where a biosensor is attached to the arm, the head, or some other part of the user, the information processing system 100 may judge an emotion of the living body by outputting information acquired from the biosensor to another terminal such as a smartphone and causing the other terminal to perform information processing.

The biosensor included in the information processing system 100 comes in contact with the surface of the living body in various ways as described above, and detects biological information. Thus, a change in contact pressure between the biosensor and the living body caused by a body motion of the living body is likely to affect a result of measurement by the biosensor. For example, the biological information acquired from the biosensor may include noise caused by the body motion of the living body. It is desired to accurately judge an emotion of the living body from biological information including such noise.

The body motion of the living body means motion forms of the living body in general, and includes, for example, motions of the living body when the information processing system 100 is attached to the wrist of the user U1, such as the user twisting his wrist, bending and stretching his fingers, and bending and stretching some of the fingers. Such a motion of the user U1 may cause a change in contact pressure between the biosensor included in the information processing system 100 and the user U1.

To improve the accuracy of information obtained by the biosensor, the information processing system 100 according to the present embodiment includes a pressure sensor that detects a body motion pressure of a region of the living body corresponding to a detection region of the biosensor. The pressure sensor detects a body motion pressure responsible for noise that reduces the accuracy of information to be detected by the biosensor. Then, using the detected body motion pressure, the information processing system 100 corrects detection data of the biosensor, thus making it possible to improve the accuracy of the detection data. Examples of information detected by the pressure sensor may include the time when a body motion pressure has been produced, a value of the body motion pressure, etc.

### (2. Internal Configuration of Information Processing System)

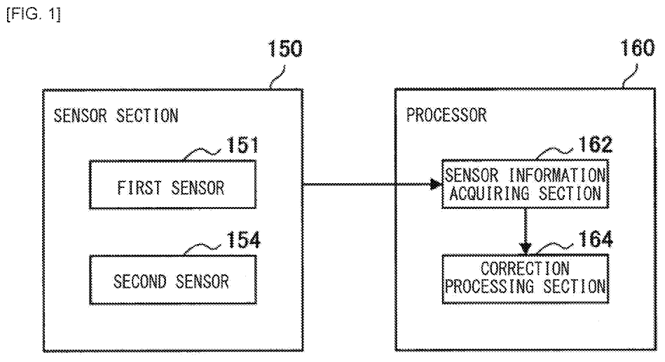

Each configuration is described with reference to FIG. 1. The information processing system 100 includes a sensor section 150 and a processor 160.

### (Sensor Section 150)

The sensor section 150 includes a first sensor 151 and a second sensor 154. The sensor section 150 has a function of acquiring information required for the processor 160 to correct first sensor information and outputting the information to the processor 160.

### (First Sensor 151)

The first sensor 151 has a function of detecting information for judging an emotion of the living body. For example, the first sensor 151 may be a sweat sensor. The sweat sensor is a sensor that detects sweat secreted from sweat glands (for example, eccrine glands) of the skin. The sweat makes the skin easy to allow passage of electricity. Therefore, the sweat sensor is able to detect a sweat by acquiring electrodermal activity (Electro Dermal Activity: EDA).

In the above description, the sweat sensor is given as an example; however, the first sensor 151 is not limited to this, and may be any type of sensor as long as it is able to detect information for judging an emotion of the living body. The sweat sensor is, for example, an example of a sensor that is attached to or brought in contact with an individual, and is an example of a biosensor having a function of detecting information (biological information) for judging an emotion of the user's living body. Other examples of the biosensor may include a pulse wave sensor, a heartbeat sensor, a blood pressure sensor, a body temperature sensor, etc. Such a biosensor makes it possible to acquire biological information related to the user's biological state. One or more of these biosensors may be provided in the information processing system 100. The biological information acquired by the biosensor is outputted to the processor 160.

### (Second Sensor 154)

The second sensor 154 has a function of detecting a body motion pressure of a region of the living body corresponding to a detection region of the first sensor 151. The second sensor 154 may be any type of sensor as long as it is a sensor that generally detects a pressure. For example, the second sensor 154 only has to be a device or something whose voltage, current, or resistance varies with pressure (such as a piezoelectric device); specifically, the second sensor 154 may be a pressure-sensitive conductive elastomer that is a conductive material mixed into a polymeric material.

The pressure-sensitive conductive elastomer deforms with a change in pressure, and thereby particles of the conductive material included in the pressure-sensitive conductive elastomer begin to come in contact with one another, which makes it possible to increase the electric conductivity in the pressure-sensitive conductive elastomer and reduce the electric resistance. On the basis of a difference in value of this electric resistance, the pressure-sensitive conductive elastomer is able to detect a pressure.

The second sensor 154 performs the detection on a region corresponding to a region subjected to the detection by the first sensor 151. The region corresponding to the region subjected to the detection by the first sensor 151 may be a region that at least a portion thereof overlaps with a region where the first sensor 151 is disposed. The second sensor 154 detects a body motion pressure of the region of the living body that at least a portion thereof overlaps with the region where the first sensor 151 is disposed, which makes it possible to more accurately correct first sensor information.

Furthermore, the region corresponding to the detection region of the first sensor 151 may be a region including the entire region where the first sensor 151 is disposed. This makes it possible for the second sensor 154 to detect the body motion pressure of the region including the detection region of the first sensor 151, and thus it is possible to more accurately detect the body motion pressure on the first sensor 151.

The detection region of the second sensor 154 is not limited to the above-described region, and may be appropriately set in accordance with the detection region of the first sensor 151. For example, the larger the detection region of the second sensor 154 is as compared with the detection region of the first sensor 151, the easier it is for the second sensor 154 to detect a body motion pressure of a region of the living body outside the detection region of the first sensor 151. Thus, in a case where the detection region of the second sensor 154 is excessively larger than the detection region of the first sensor 151, there is a possibility of a decrease in the accuracy of detection of the body motion pressure on the first sensor 151. Therefore, the detection region of the second sensor 154 may be appropriately set in accordance with, for example, a disposition relationship between the first sensor 151 and the second sensor 154 or the area of the region.

Moreover, the region corresponding to the detection region of the first sensor 151 may be a region near the region where the first sensor 151 is disposed, and does not necessarily have to include a portion that overlaps with the region where the first sensor 151 is disposed. By detecting a body motion pressure of the region near the region where the first sensor 151 is disposed, it becomes possible to approximately acquire a body motion pressure on the region subjected to the detection by the first sensor 151, and therefore it is possible to correct first sensor information.

Furthermore, the second sensor 154 may be calibrated at predetermined timing. By the second sensor 154 being calibrated, it becomes possible to more accurately detect a body motion pressure of the living body. For example, when the user has worn the information processing system 100, the second sensor 154 may be calibrated. Since the user has worn the information processing system 100, a contact pressure between the living body and the information processing system 100 begins to be generated. To detect a body motion pressure of the living body, merely a contact pressure between the living body at rest and the information processing system 100 (also referred to as a static pressure) may become an unnecessary detected pressure. Thus, by performing the calibration when the user has worn the information processing system 100, it becomes possible for the second sensor 154 to more accurately detect a body motion pressure excluding the static pressure.

### (Processor 160)

The processor 160 includes a sensor information acquiring section 162 and a correction processing section 164. The processor 160 has a function of acquiring first sensor information and second sensor information from the sensor section 150 and correcting the first sensor information.

### (Sensor Information Acquiring Section 162)

The sensor information acquiring section 162 acquires first sensor information and second sensor information from the first sensor 151 and the second sensor 154, respectively. The first sensor information is information for judging an emotion of the living body. For example, in a case where the first sensor 151 is a sweat sensor, the first sensor information may include information regarding the time when development of sweat has started, information regarding the sweat rate, etc. The second sensor information is information regarding a body motion pressure of the living body. For example, the second sensor information may include body motion pressure information such as the start and end times of a body motion pressure detected by the second sensor 154 when the living body has made a body motion, the duration of the body motion pressure, or a value of the body motion pressure.

### (Correction Processing Section 164)

The correction processing section 164 has a function of correcting first sensor information on the basis of the first sensor information and second sensor information that the sensor information acquiring section 162 has acquired. For example, in a case where the first sensor 151 is a sweat sensor, the correction processing section 164 has a function of removing noise, etc. included in information obtained by the sweat sensor, thereby correcting the first sensor information. Specifically, the correction processing section 164 performs correction processing of identifying noise included in the first sensor information on the basis of body motion pressure information such as the start and end times of a pressure or a value of the pressure acquired by a pressure sensor that detects a body motion pressure of the living body detected by the second sensor 154 and removing the noise from the first sensor information.

For example, with respect to a signal acquired by the first sensor 151, the correction processing section 164 may determine an increase or decrease in value of a pressure detected by the second sensor 154 to be noise and remove it. Furthermore, for example, in a case where a change in the trend of a signal acquired by the first sensor 151 is shown at the time when the second sensor 154 has detected a body motion pressure of the living body, a body motion pressure while the second sensor 154 has detected the body motion pressure of the living body may be removed from the signal acquired by the first sensor 151.

### (3. External Configuration of Information Processing System)

### (3.1. Outline of Configuration)

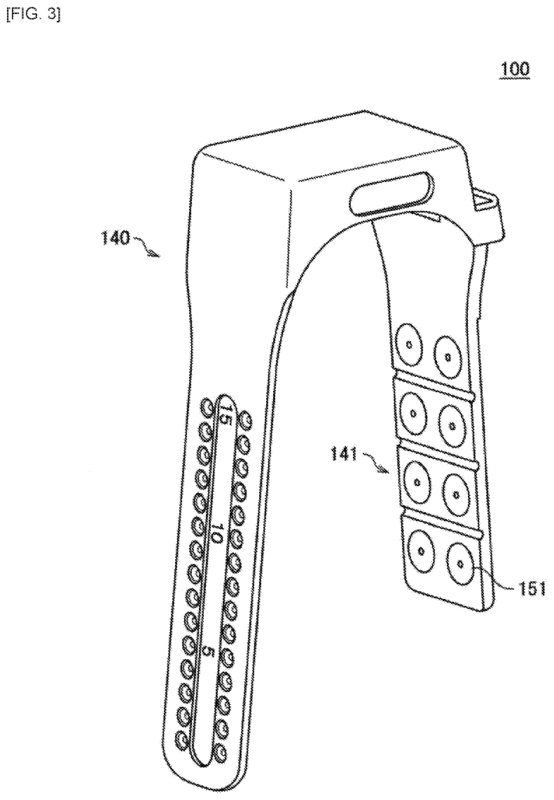

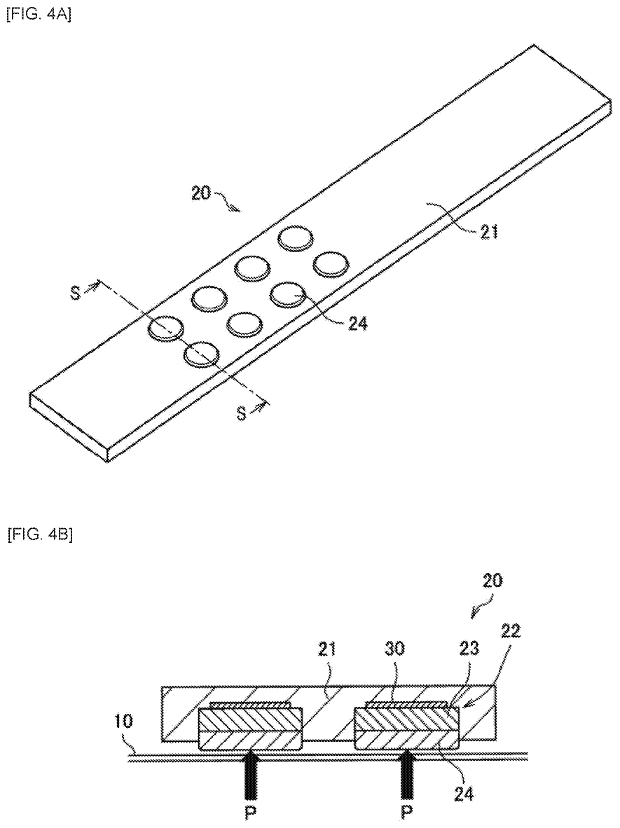

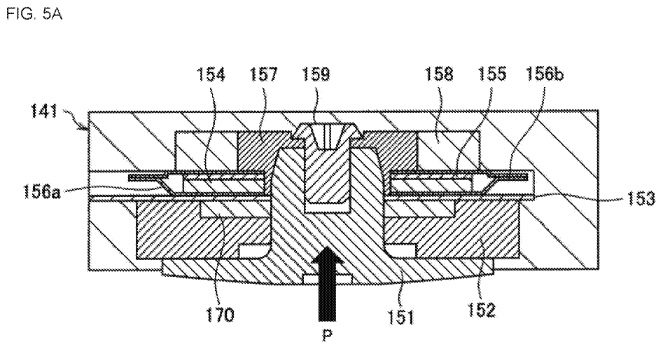

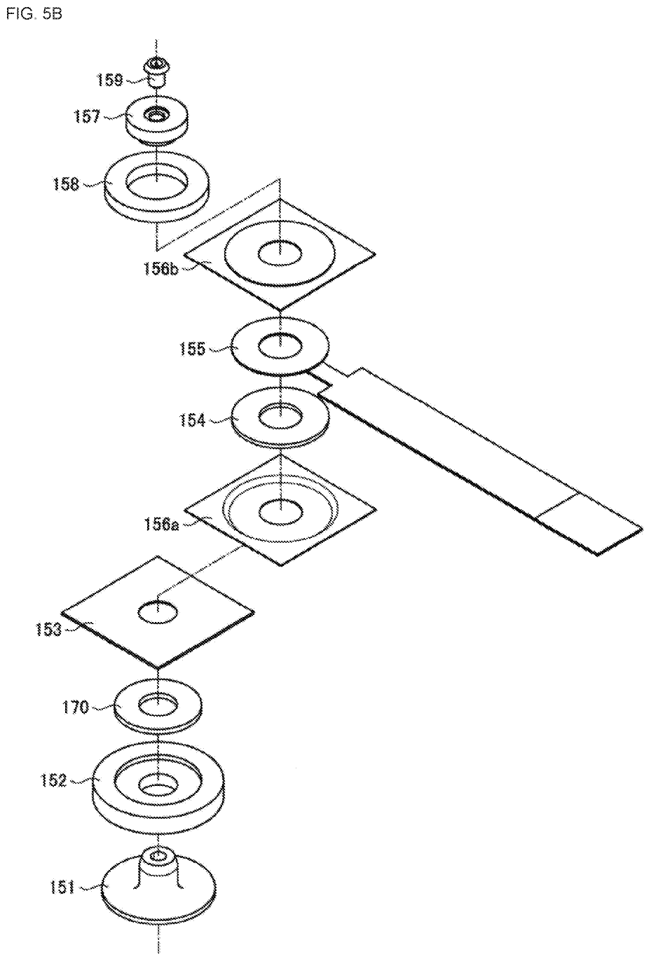

Subsequently, an outline of an external configuration of the information processing system 100 is described with reference to FIGS. 3 to 5. FIG. 3 is a diagram illustrating an example of the appearance of the information processing system 100. FIGS. 4A and 4B are diagrams illustrating a configuration of a sensor section included in the information processing system 100. FIGS. 5A and 5B are diagrams illustrating the configuration of the sensor section illustrated in FIGS. 4A and 4B in more detail.

First, an example of the configuration of the information processing system 100 is described with reference to FIG. 3. The information processing system 100 includes a wristwatch-type biosensor module 140, and a biosensor 151 is built into a wristband 141 to be exposed on the surface of the wristband 141. The wristband 141 has a function of supporting the biosensor 151, etc. The wristband 141 has a shape extending in one direction, and is attached by being wrapped around a living body like a wristwatch. The wristband 141 may include rubber or leather, or may include organic resin or the like. On the living body side of the wristband 141, a pair of the biosensors 151 is provided at equal intervals in an extending direction of the wristband. The shape of an exposed portion of each biosensor 151 may be a circular shape. In this example, the biosensor 151 is circular; however, the shape of the biosensor 151 is not limited, and may be the shape of, for example, an ellipse, a rectangle, or a polygon.

Furthermore, the number of the biosensors 151 provided in the wristband 141 is also not particularly limited, and one or more biosensors 151 only have to be provided. Between the biosensor 151 and the wristband 141, a different sensor from the biosensor 151 is provided for detecting deformation of the wristband 141, a force applied to the wristband 141, and a change in the shape of the wristband 141. For example, a pressure sensor is provided between an exposed surface of the biosensor 151 and the wristband 141. This pressure sensor makes it possible for the information processing system 100 attached to the user's wrist to detect a change in a body motion pressure in accordance with the motion of the wrist.

Next, how the biosensor 151 and the pressure sensor function is described with a schematic diagram representing the biosensor 151 provided in the wristband 141 with reference to FIGS. 4A and 4B.

A wristband 21 is provided with a pair of sensor sections 22 at equal intervals in an extending direction of the wristband 21. FIG. 4B is a cross-sectional view along a line S-S illustrated in FIG. 4A, and illustrates a state of the wristband 21 attached around the surface of a living body 10. The sensor section 22 is built into the wristband 21 attached to the surface of the living body 10. The sensor section 22 and the wristband 21 have a three-layered stacked structure, and a biosensor 24, a pressure sensor 30, and the wristband 21 are disposed to be stacked in layers in this order from the living body side. A region where the pressure sensor 30 is disposed overlaps with a region where the biosensor 24 is disposed, and the pressure sensor 30 is disposed right above the biosensor 24 in a direction on the side opposite to the living body side.

A deformable member 23 is disposed between the biosensor 24 and the pressure sensor 30. The deformable member 23 includes a polymeric material, and deforms with pressure and is restored to its original shape when released from the pressure. Specifically, the deformable member 23 may include rubber, or may include organic resin or the like. For example, the deformable member 23 may include silicone rubber. The deformable member 23 may include a material having a larger amount of displacement than the wristband 21 in a case where they are pressed with the same pressure.

For example, the deformable member 23 may include a material having a lower durometer hardness than the wristband 21. It is to be noted that the durometer hardness in the present embodiment complies with the durometer hardness (type A), JIS K 6253. Specifically, the deformable member 23 may have a durometer hardness of 20 or lower, and the wristband 21 may have a durometer hardness of higher than 20 and lower than 90. For example, the deformable member 23 may have a durometer hardness of 7°, and the wristband 21 may have a durometer hardness of 40°.

In the information processing system having the above-described configuration, a sensor electrode of the biosensor 24 is displaced in a direction of an arrow illustrated in FIG. 4B by a pressing force P from the side of a mounting surface typified by, for example, the skin of the living body. This displacement is generated over the entire wristband 21; however, the deformable member 23 having a lower hardness is more largely displaced because of a hardness difference between the main body of the wristband 21, using a member having a higher hardness than the deformable member 23, and the deformable member 23. A force generated as a reaction force to this compressive deformation of the deformable member 23 is transmitted to the pressure sensor 30, which makes it possible for the biosensor 24 to detect a pressure applied to the sensor electrode.

It is to be noted that the deformation of the wristband 21 caused by being pressed from an external environment on the side opposite to the living body because of this durometer hardness difference is extremely small. Thus, the influence on the pressure sensor 30 built into the wristband 21 from the external environment on the side opposite to the living body is negligible. According to this configuration, it is possible to further improve the accuracy of pressure detected by the second sensor 154.

In the detection of a body motion pressure by the pressure sensor 30, depending on the pressing force P to press the biosensor 24 against the surface of the living body or the composition of the living body, etc., there may be a case where a pressing surface between the surface of the living body 10 and the biosensor 24 is uneven. In such a case, the deformable member 23 is deformed in accordance with a surface shape of the living body 10 by the pressing, making it possible to obtain a state in which the surface of the living body and the pressing surface of the biosensor 24 are parallel to each other. Thus, the pressing surface and the surface of the living body become parallel to each other, which makes it possible to accurately transmit the pressing force of the surface of the living body to the pressure sensor 30, and therefore it is possible to improve the detection accuracy of the pressure sensor 30.

### (3.2. Details of Configuration)

Subsequently, an example of the detailed configuration of the information processing system 100 according to the present embodiment is described with reference to FIGS. 5A and 5B. FIG. 5A is a cross-sectional view of, of a pair of the sensor sections provided in the wristband 141 of the information processing system 100 illustrated in FIG. 3, one sensor section cut in a direction of the short side of the wristband 141. FIG. 5B is an exploded perspective view of the structure illustrated in FIG. 5A.

As seen in FIG. 5A, the sensor section is built into the wristband 141, and the projection-shaped biosensor 151 is provided on the side closest to the living body. The biosensor 151 is disposed to be exposed on the living-body-side surface of the wristband 141. In the wristband 141 illustrated in FIGS. 5A and 5B, the biosensor 151 is configured to be exposed on the surface of the wristband 141, and therefore is able to come in contact with the surface of the living body and acquire information for judging an emotion of the living body. The wristband 141 has various components stacked in its inside. In the following description, a direction in which the components inside the wristband 141 are stacked is referred to as an up-down direction; a direction in which the living body is present when the wristband 141 is attached to the living body is referred to as a downward direction; and a direction opposite to the downward direction is referred to as an upward direction.

The biosensor 151 is formed into a projection shape that projects in the upward direction from a contact surface of the wristband 141 with the living body. A projection portion of the projection shape is formed to project straight upward on the center of the biosensor 151 toward a surface of the wristband 141 on the side opposite to the living body. Over a portion of the biosensor 151 from a component on the contact surface to the finishing part of the projection portion, various circular components are provided on the same central axis as the projection shape.

A deformable member 152 is provided on top of a contact portion of the biosensor 151. The deformable member 152 is formed to have the larger area than a region of the biosensor 151 in contact with the living body. The thickness of the deformable member 152 in a stacked direction is substantially half the thickness of the projection portion of the biosensor 151. In a portion of the upper part of the deformable member 152, a conductive resin 170 is put to be embedded on the side of the biosensor, and is disposed in contact with the biosensor 151. The conductive resin 170 has a radius of substantially half the length of the radius of the deformable member 152 that extends in a circular fashion with the projection portion of the biosensor 151 as the central axis, and is built into the deformable member 152.

On top of respective contact portions of the deformable member 152 and the conductive resin 170, a biosensor wiring line 153 that stretches over the deformable member 152 and the conductive resin 170 is disposed. That is, the sensor electrode of the projection-shaped biosensor 151 is fixed to the biosensor wiring line 153 through the deformable member 152 stacked on the biosensor 151 by the conductive resin 170. The biosensor wiring line 153 is formed into a square shape with the central axis of the biosensor 151 as the center.

Polyimide sheets 156a and 156b are disposed on top of the biosensor wiring line 153, and a pressure sensor 154 and a pressure sensor section wiring line 155 are disposed to be held between the polyimide sheets 156a and 156b. On top of a contact portion of the pressure sensor 154, the pressure sensor section wiring line 155 is disposed. At this time, the polyimide sheets 156a and 156b have a function of fixating the pressure sensor 154. A pressure sensor section including the polyimide sheets 156a and 156b, the pressure sensor 154, and the pressure sensor section wiring line 155 has a hollow structure having no direct contact with the projection portion of the biosensor 151. A sleeve 157 is fitted into the hollow structure, and thus the pressure sensor section is in contact with the biosensor 151 through the sleeve 157. Furthermore, as with the biosensor wiring line 153, the polyimide sheets 156a and 156b are formed into a square shape with the central axis of the biosensor 151 as the center.

On top of respective contact portions and hollow structures of the polyimide sheets 156a and 156b holding the pressure sensor 154 and the pressure sensor section wiring line 155 between them, the sleeve 157 is disposed on the side of the biosensor 151, and, in a direction opposite to a direction in which the biosensor 151 is provided, a washer 158 is disposed adjacent to the sleeve 157. The sleeve 157 and the washer 158 may include synthetic resin or the like, and the sleeve 157 may include polycarbonate resin. Furthermore, the washer 158 may include PBT (polybutylene terephthalate) resin. The washer 158 has a function of maintaining flatness of the pressure sensor section, and is attached to the sleeve 157.

Moreover, the pressure sensor section is stacked on the biosensor wiring line 153, and is fixed to the top of the sensor electrode of the biosensor 151 through the sleeve 157 by a screw 59.

The above-described configuration makes it possible for the wristband-type information processing system including the biosensor to effectively detect a pressure in a limited direction at an intended point upon detecting, by the pressure sensor, pressing performed on a wristband mounting surface side.

FIG. 5B is an exploded view of the information processing system illustrated in FIG. 5A. As seen in FIG. 5B, the components with a hole in the center are disposed to be stacked on the projection portion of the biosensor 151 having a projection shape. On top of the biosensor 151, the deformable member 152 having different thicknesses is stacked in a circular radial direction. The conductive resin 170 is disposed on a thin portion of the deformable member 152. A difference in thickness between the thin portion and a thick portion of the deformable member 152 is equal to the thickness of the conductive resin 170, and the conductive resin 170 is fitted into the deformable member 152. Thus, the deformable member 152 is integrated with the conductive resin 170, and has a uniform thickness.

On top of the respective contact portions of the deformable member 152 and the conductive resin 170 that form the uniform thickness, the biosensor wiring line 153 having the larger region than the deformable member 152 and the conductive resin 170 is stacked. The polyimide sheet 156a having the region equal to the biosensor wiring line 153 is stacked on the biosensor wiring line 153. The polyimide sheet 156a has a tapered shape that extends in a circle outer diameter direction from its circular lowermost part, and has a shape connecting to its square-shaped uppermost part through the tapered shape. The pressure sensor 154 and the pressure sensor section wiring line 155 are stacked between the uppermost part and the lowermost part of the polyimide sheet 156a. The polyimide sheet 156b is further stacked on top of respective contact portions of the pressure sensor 154 and the pressure sensor section wiring line 155, and the pressure sensor 154 and the pressure sensor section wiring line 155 are enveloped by the polyimide sheets 156a and 156b.

On top of the contact portion of the polyimide sheet 156b, the washer 158 with a larger hole than the project portion of the biosensor 151 in the center is stacked, and the sleeve 157 is fitted into the hole of the washer 158. The sleeve 157 has a tapped hole in its center, and is fixed by the screw 59.

The above is a configuration of the information processing system into which the biosensor 151 and the pressure sensor 154 are built.

### (4. Operation Flow of Information Processing System)

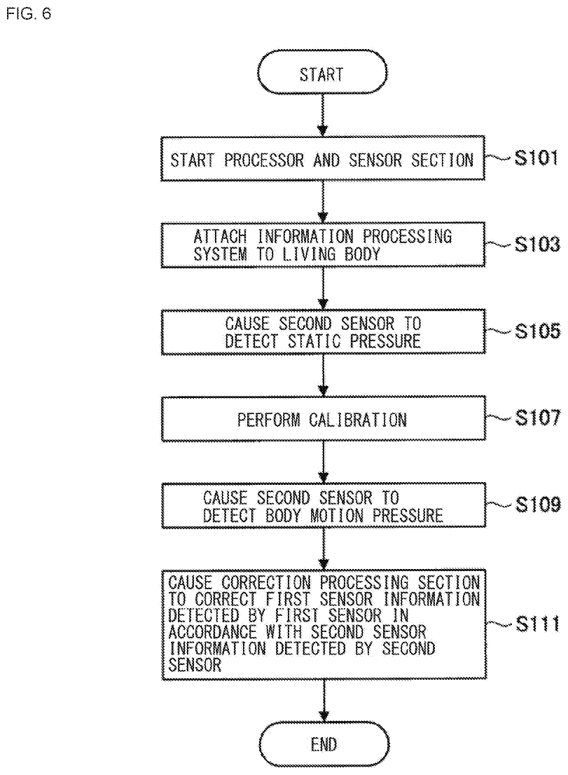

Subsequently, the operation flow of each of the above-described components is described with reference to FIG. 6.

First, the sensor section 150 and the processor 160 of the information processing system 100 are caused to start, for example, by depression of the power button by a user (S101).

Next, the information processing system 100 is attached to a living body such as the user (S103). When the information processing system 100 has been attached to the living body, a static pressure when the information processing system 100 has been attached to the living body is detected by the second sensor 154 included in the information processing system 100 (S105). The static pressure indicates the living body is at rest, and there is no temporal change in pressing force applied to the information processing system 100. For example, the static pressure means a pressure given from the living body at the time when the living body is at rest.

On the basis of the detected static pressure, the information processing system 100 performs calibration of the second sensor 154 (S107). By performing the calibration, it becomes possible for the information processing system 100 to determine a reference point of body motion pressure detected by the second sensor 154 and detect the magnitude of a body motion pressure on the basis of a pressure difference from the reference point. Here, there is described an example where the timing to detect the static pressure is the time when the information processing system 100 has been attached to the living body; however, it is not limited to this example, and may be a predetermined timing.

For example, the timing to detect the static pressure may be the time when the user refastens the wristband 41 to prevent the information processing system 100 from moving from an attachment position on the living body. As another example, the detection of the static pressure and the calibration of the second sensor 154 may be performed in a case where the temperature changes with the passage of a time the information processing system 100 is attached to the user, and an intended body motion pressure is not obtained.

After the calibration has been performed, a body motion pressure of the living body is detected by means of the second sensor 154 (S109).

The correction processing section 164 of the information processing system 100 corrects first sensor information detected by the biosensor 151 in accordance with the second sensor information detected by the second sensor 154 (S119). In a case where the first sensor information has been corrected by the correction processing section 164, the information processing system 100 ends the operation.

### (5. Hardware Configuration Example)

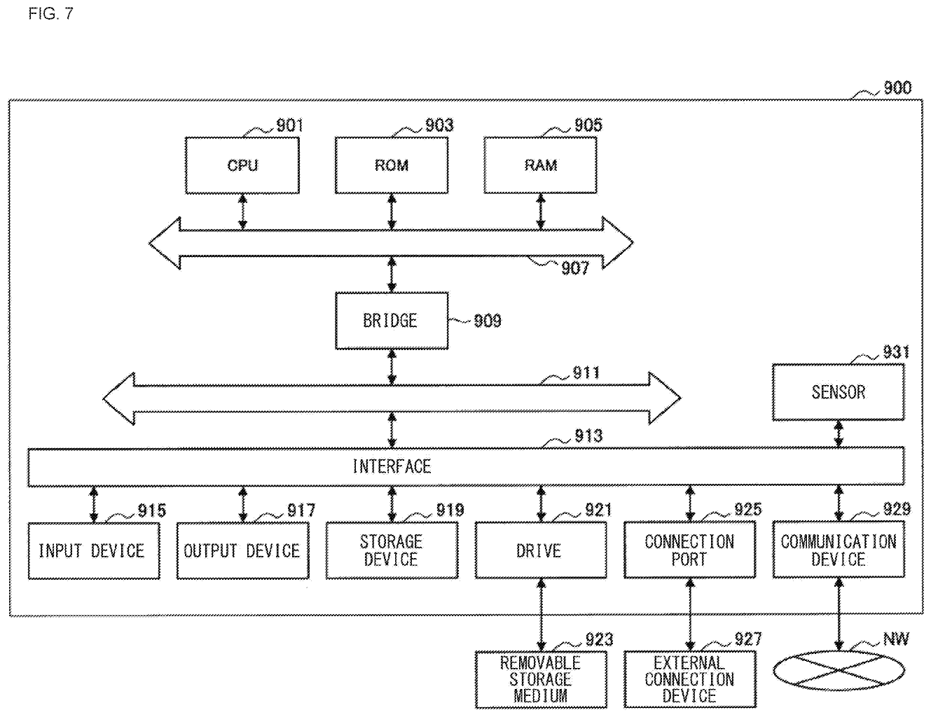

Subsequently, a hardware configuration of an information processing apparatus 900 that may perform the information processing by the information processing system according to the embodiment of the present disclosure is described with reference to FIG. 7. FIG. 7 is a block diagram illustrating a hardware configuration example of the information processing apparatus 900.

The information processing apparatus 900 includes a CPU (Central Processing Unit) 901, a ROM (Read Only Memory) 903, and a RAM (Random Access Memory) 905.

Furthermore, the information processing apparatus 900 may include a host bus 907, a bridge 909, an external bus 911, an interface 913, an input device 915, an output device 917, a storage device 919, a drive 921, a connection port 925, a communication device 929, and a sensor 931. The information processing apparatus 900 may include a processing circuit, such as a DSP (Digital Signal Processor) or an ASIC (Application Specific Integrated Circuit), instead of or together with the CPU 901.

The CPU 901 serves as an arithmetic processing unit and a control device, and controls the entire operation or some of the operation in the information processing apparatus 900 in accordance with various programs recorded in the ROM 903, the RAM 905, the storage device 919, or a removable recording medium 923. The ROM 903 stores therein a program, an operation parameter, etc. that the CPU 901 uses. The RAM 905 temporarily stores therein a program used when the CPU 901 executes, a parameter that is appropriately changed through the execution of the program, etc. For example, the CPU 901, the ROM 903, and the RAM 905 may realize the function of the processor 160 in the above-described embodiment. The CPU 901, the ROM 903, and the RAM are coupled to one another by the host bus 907 including an internal bus such as a CPU bus. Furthermore, the host bus 907 is coupled to the external bus 911 such as a PCI (Peripheral Component Interconnect/Interface) bus through the bridge 909.

The input device 915 is a device operated by a user, for example, such as a mouse, a keyboard, a touch panel, a button, a switch, or a lever. For example, the input device 915 may be a remote control device using infrared rays or other radio waves, or may be an external connection device 927 such as a mobile phone compatible with the operation of the information processing apparatus 900. The input device 915 includes an input control circuit that generates an input signal on the basis of information inputted by the user and outputs the input signal to the CPU 901. The user operates this input device 915, and thereby inputs various data or issues an instruction for a processing operation to the information processing apparatus 900.

The output device 917 includes a device that is able to notify the user of acquired information visually or auditorily. The output device 917 may be, for example, a display device such as an LCD (Liquid Crystal Display), a PDP (Plasma Display Panel), or an OELD (Organic Electro-Luminescence Display), a voice output device such as a speaker and a headphone, and a printer device, etc. The output device 917 outputs a result obtained through processing by the information processing apparatus 900 as a projected image such as a text or an image, or outputs the result as a voice such as a speech or a sound.

The storage device 919 is a device for data storage configured as an example of storage of the information processing apparatus 900. The storage device 919 includes, for example, a magnetic storage device such as an HDD (Hard Disk Drive), a semiconductor storage device, an optical storage device, a magneto-optical storage device, etc. This storage device 919 stores therein a program and various data that the CPU 901 executes, various data acquired from the outside, etc.

The drive 921 is a reader/writer for the removable recording medium 923 such as a magnetic disk, an optical disk, a magneto-optical disk, or a semiconductor memory, and is built into or externally attached to the information processing apparatus 900. The drive 921 reads out information recorded in the mounted removable recording medium 923, and outputs the information to the RAM 905. Furthermore, the drive 921 writes a document in the mounted removable recording medium 923. It is to be noted that at least either the storage device 919 or the drive 921 and the removable recording medium 923 may realize a storage function of the processor 160 according to the above-described embodiment.

The connection port 925 is a port for coupling a device directly to the information processing apparatus 900. The connection port 925 may be, for example, a USB (Universal Serial Bus) port, an IEEE1394 port, an SCSI (Small Computer System Interface) port, etc. Furthermore, the connection port 925 may be an RS-232C port, an optical audio terminal, an HDMI (Registered Trademark) (High-Definition Multimedia Interface) port, etc. By coupling the external connection device 927 to the connection port 925, it becomes possible to exchange various data between the information processing apparatus 900 and the external connection device 927.

The communication device 929 is, for example, a communication interface including a communication device, etc. for coupling the information processing apparatus 900 to a communication network NW. The communication device 929 may be, for example, a communication card for wired or wireless LAN (Local Area Network), Bluetooth (Registered Trademark), or WUSB (Wireless USB). Furthermore, the communication device 929 may be a router for ADSL (Asymmetric Digital Subscriber Line) or a modem, etc. for various communications. For example, the communication device 929 transmits and receives a signal, etc. using a predetermined protocol such as TCP/IP with the Internet and another communication device. Moreover, the communication network NW coupled to the communication device 929 is a network coupled by wired or wireless connection, and is, for example, the Internet, a Home LAN, infrared communication, radio wave communication, satellite communication, or the like. It is to be noted that at least either the connection port 925 or the communication device 929 may realize a communication function, etc. between the sensor section 150 and the processor 160 according to the above-described embodiment.

As above, the preferred embodiment of the present disclosure has been described in detail with reference to the accompanying drawings; however, the technical scope of the present disclosure is not limited to these examples. It is apparent that those having ordinary skill in the technical field of the present disclosure could easily arrive at various modified examples or revised examples within the meaning of the technical concept described in claims, and it is understood that these also should naturally fall under the technical scope of the present disclosure.

For example, in the above-described embodiment, the information processing system 100 is described as the wristwatch-type information processing system; however, a technique according to the present disclosure is not limited to this example. For example, the information processing system 100 may be a head-mounted information processing system.

Furthermore, the effects described in the present specification are only explanatory or exemplary and not limitative. That is, a technique according to the present disclosure may achieve other effects that should be understood from description of the present specification by those skilled in the art in addition to the above-described effects or instead of the above-described effects.

It is to be noted that the following configurations also fall within the technical scope of the present disclosure.

(1)

An information processing system including:

a sensor section including
a first sensor that detects information for judging an emotion of a living body, and
a second sensor that detects a body motion pressure of a region of the living body corresponding to a detection region of the first sensor; and

a correction processing section that corrects first sensor information obtained by the first sensor on the basis of second sensor information obtained by the second sensor.

(2)

The information processing system according to (1), further including a support part to be attached along the living body,

in which the sensor section is included in the support part.

(3)

The information processing system according to (2), in which

the support part has a shape extending in one direction, and

the support part is to be attached by wrapping the extending shape around the living body.

(4)

The information processing system according to (2) or (3), in which the sensor section with the first sensor and the second sensor stacked in this order from the side of the living body is built into a predetermined portion of the support part.

(5)

The information processing system according to any one of (2) to (4), in which the first sensor is exposed on a surface of the support part.

(6)

The information processing system according to any one of (1) to (4), in which a deformable member is disposed between the first sensor and the second sensor.

(7)

The information processing system according to any one of (2) to (6), in which

the deformable member and the support part include a polymeric material, and

the deformable member has a durometer hardness lower than a durometer hardness of the support part.

(8)

The information processing system according to any one of (1) to (7), in which the second sensor is calibrated at predetermined timing.

(9)

The information processing system according to any one of (1) to (8), in which the region of which the body motion pressure is to be detected by the second sensor is a region that at least a portion thereof overlaps with a region where the first sensor is disposed.

(10)

The information processing system according to any one of (1) to (8), in which the region of which the body motion pressure is to be detected by the second sensor is a region near a region where the first sensor is disposed.

(11)

The information processing system according to any one of (1) to (9), in which

the second sensor is provided right above the first sensor, where a side of the living body in the information processing system is directed in a downward direction and a direction of a side opposite to the living body is an upward direction.

(12)

The information processing system according to any one of (1) to (11), in which the second sensor is a pressure-sensitive conductive elastomer.

(13)

The information processing system according to any one of (1) to (12), in which the first sensor is a sweat sensor.

(14)

The information processing system according to any one of (2) to (13), in which

the deformable member has a durometer hardness of 20 or lower, and

the support part has a durometer hardness of higher than 20 and lower than 90.

(15)

An information processing apparatus including:

a sensor section including
a first sensor that detects information for judging an emotion of a living body, and
a second sensor that detects a body motion pressure of a region of the living body corresponding to a detection region of the first sensor; and

a correction processing section that corrects first sensor information obtained by the first sensor on the basis of second sensor information obtained by the second sensor.

(16)

An information processing method implemented by a processor, the information processing method including:

acquiring respective pieces of information detected by a first sensor and a second sensor, the first sensor detecting information for judging an emotion of a living body, the second sensor detecting a body motion pressure of a region of the living body corresponding to a detection region of the first sensor; and

correcting first sensor information obtained by the first sensor on the basis of second sensor information obtained by the second sensor.

### REFERENCE SIGNS LIST

100 information processing system
140 biosensor module
141 wristband
150 sensor section
151 first sensor
152 deformable member
153 biosensor wiring line
154 second sensor
155 pressure sensor section wiring line
156a, 156b polyimide sheet
157 sleeve
158 washer
160 processor
162 sensor information acquiring section
164 correction processing section
170 conductive resin

1. An information processing system comprising:
a sensor section including
a first sensor that detects information for judging an emotion of a living body, and
a second sensor that detects a body motion pressure of a region of the living body corresponding to a detection region of the first sensor; and
a correction processing section that corrects first sensor information obtained by the first sensor on a basis of second sensor information obtained by the second sensor.

2. The information processing system according to claim 1, further comprising a support part to be attached along the living body,
wherein the sensor section is included in the support part.

3. The information processing system according to claim 2, wherein
the support part has a shape extending in one direction, and
the support part is to be attached by wrapping the extending shape around the living body.

4. The information processing system according to claim 3, wherein the sensor section with the first sensor and the second sensor stacked in this order from a side of the living body is built into a predetermined portion of the support part.

5. The information processing system according to claim 4, wherein the first sensor is exposed on a surface of the support part.

6. The information processing system according to claim 4, wherein a deformable member is disposed between the first sensor and the second sensor.

7. The information processing system according to claim 6, wherein
the deformable member and the support part include a polymeric material, and
the deformable member has a durometer hardness lower than a durometer hardness of the support part.

8. The information processing system according to claim 1, wherein the second sensor is calibrated at predetermined timing.

9. The information processing system according to claim 1, wherein the region of which the body motion pressure is to be detected by the second sensor is a region that at least a portion thereof overlaps with a region where the first sensor is disposed.

10. The information processing system according to claim 1, wherein the region of which the body motion pressure is to be detected by the second sensor is a region near a region where the first sensor is disposed.

11. The information processing system according to claim 9, wherein
the second sensor is provided right above the first sensor, where a side of the living body in the information processing system is directed in a downward direction and a direction of a side opposite to the living body is an upward direction.

12. The information processing system according to claim 1, wherein the second sensor is a pressure-sensitive conductive elastomer.

13. The information processing system according to claim 1, wherein the first sensor is a sweat sensor.

14. The information processing system according to claim 7, wherein
the deformable member has a durometer hardness of 20 or lower, and
the support part has a durometer hardness of higher than 20 and lower than 90.

15. An information processing apparatus comprising:
a sensor section including
a first sensor that detects information for judging an emotion of a living body, and
a second sensor that detects a body motion pressure of a region of the living body corresponding to a detection region of the first sensor; and
a correction processing section that corrects first sensor information obtained by the first sensor on a basis of second sensor information obtained by the second sensor.

16. An information processing method implemented by a processor, the information processing method comprising:
acquiring respective pieces of information detected by a first sensor and a second sensor, the first sensor detecting information for judging an emotion of a living body, the second sensor detecting a body motion pressure of a region of the living body corresponding to a detection region of the first sensor; and
correcting first sensor information obtained by the first sensor on a basis of second sensor information obtained by the second sensor.
